(19) United States
(12) Patent Application Publication
(10) Pub. No.: US 2003/0036519 A1
(43) Pub. Date: Feb. 20, 2003

(54) STABLE ALTERATION OF PRE-MRNA SPLICING PATTERNS BY MODIFIED RNAS

(76) Inventors: RYSZARD KOLE, CHAPEL HILL, NC (US); DANIEL SCHUMPERLI, BUEHLSTR (CH); HALINA SIERAKOOWSKA, CARRBORO, NC (US); DANIEL SUTER, SUNNHALE (CH)

Correspondence Address: MYERS BIGEL SIBLEY & SAJOVEC, PO BOX 37428, RALEIGH, NC, 27627, US

(21) Appl. No.: 09/295,176

(22) Filed: Apr. 20, 1999

Related U.S. Application Data
(60) Provisional application No. 60/082,510, filed on Apr. 21, 1998.

(51) Int. Cl. A61K 48/00; C07H 21/02

(52) U.S. Cl. 514/44; 536/23.1

(57) ABSTRACT

The present invention provides a method of upregulating expression of a protein of interest (e.g., a native protein) in a cell, the cell containing a DNA encoding the protein, which DNA contains a mutation that causes downregulation of the protein by aberrant splicing in a pre-mRNA, wherein the DNA encodes the pre-mRNA; wherein the pre-mRNA contains a native intron having a first set of splice elements, which native intron is removed by splicing when the mutation is absent to produce a first mRNA encoding the protein; and wherein the pre-mRNA further contains an aberrant intron different from the native intron having a second set of splice elements, which aberrant intron is removed by splicing when the mutation is present to produce an aberrant second mRNA different from the first mRNA. The method comprises administering to the cell a gene transfer vector a heterologous oligonucleotide in the cell, the heterologous oligonucleotide comprising a nuclear localization element joined to an antisense oligonucleotide, which antisense oligonucleotide hybridizes to the pre-mRNA in the nucleus of the cell to create a duplex thereof under conditions which permit splicing, and wherein the antisense oligonucleotide blocks a member of the aberrant second set of splice elements so that the native intron is removed by splicing and the protein of interest is produced. Vectors and oligonucleotides useful for carrying out the method are also disclosed.

### RELATED APPLICATIONS

This application claims priority from R. Kole et al., United States Provisional Application No. 60/082,510, filed Apr. 21, 1998, the disclosure of which is incorporated by reference herein in its entirety.

This invention was made with government support under grant number IR-1HL51940-1 from the National Institutes of Health. The Government has certain rights to this invention.

### FIELD OF THE INVENTION

This invention relates to methods of combating aberrant splicing of pre-mRNA molecules and upregulating gene expression, along with products useful therefore.

### BACKGROUND OF THE INVENTION

Gene therapy appears to be the most promising treatment for genetic disorders (reviewed in 1). It is usually understood as either the replacement of a defective gene with the correct one or expression of a transgene whose product supplants its defective counterpart. These forms of gene therapy have been tested in animal models and in the clinic, for example, in treatment of adenosine deaminase deficiency (1, 2), cystic fibrosis (3), and other genetic disorders (4, 5). Although, in principle, gene therapy should be applicable to any gene-based disorder, the difficulties with vectors suitable for efficient delivery of large transgenes or providing sustained expression of the transfected genes in a tissue-specific, properly regulated manner (6, 7) limit its clinical applicability. Regulated expression is especially important in gene therapy for correction of tightly regulated genes such as β-globin in sickle cell anemia or thalassemia. Expression of the β-globin transgene is useful only if it occurs in concert with the α-globin genes in erythroid cells. Although the β-globin gene is small, its regulated expression is difficult to achieve since it is controlled by a large locus control region (LCR). Vectors capable of accommodating large fragments of DNA are not yet available, while truncated constructs, in spite of significant progress, do not provide the desired levels and specificity of expression (8-10).

In addition to gene replacement, gene therapy may also be accomplished by manipulation of gene structure and expression. It has recently been shown in model cell culture systems that double stranded chimeric RNA-DNA oligonucleotides may induce site specifc removal from the human β-globin gene of the mutation responsible for sickle cell anemia (11). Clinically relevant alteration of globin gene expression can be also achieved by relatively simple pharmacological treatments. For example, hydroxyurea or butyric acid and its derivatives induce the expression of fetal hemoglobin which partially compensates for the lack of correct β-globin expression in sickle cell anemia or thalassemia. These treatments were successful in clinical trials (12-15).

U.S. Pat. No. 5, 665,593 to Kole et al. shows that antisense oligonucleotides restores the activity of thalassemic β-globin genes carrying mutations that cause defects in pre-mRNA splicing. Oligonucleotides targeted to the aberrant splice sites generated by the thalassemic mutations in intron 2 of the β-globin gene: IVS2-654, -705, and -745 (16, 17, unpublished data), blocked the aberrant splice sites and restored the correct splicing pattern by forcing the splicing machinery to reselect the existing correct splice sites. The correction of splicing was accompanied by translation of the resultant β-globin mRNA into full length β-globin protein. If the same results were achieved in the erythroblasts of a thalassemic patient, a more balanced synthesis of α-and β-globin would be restored and the clinical symptoms of thalassemia ameliorated. Note that in patients, the antisense oligonucleotides would have restored correct splicing of pre-mRNA, properly transcribed from the β-globin gene in its natural chromosomal environment. This precludes the possibility of overexpression of β-globin mRNA, an important consideration in treatment of hemoglobinopathies. However, a significant drawback of this approach stems from the fact that the oligonucleotides do not remove the mutation and would therefore require periodic administrations.

### SUMMARY OF THE INVENTION

A first aspect of the present invention is a method of upregulating expression of a protein of interest (e.g., a native protein) in a cell, the cell containing a DNA encoding the protein, which DNA contains a mutation that causes downregulation of the protein by aberrant splicing in a pre-mRNA, wherein the DNA encodes the pre-mRNA; wherein the pre-mRNA contains a native intron having a first set of splice elements, which native intron is removed by splicing when the mutation is absent to produce a first mRNA encoding the protein; and wherein the pre-mRNA further contains an aberrant intron different from the native intron having a second set of splice elements, which aberrant intron is removed by splicing when the mutation is present to produce an aberrant second mRNA different from the first mRNA. The method comprises administering (in vivo or in vitro) to the cell a gene transfer vector (e.g., a viral vector) that expresses a heterologous RNA in the cell (e.g., small nuclear RNA), the heterologous RNA comprising a nuclear localization element joined to an antisense oligonucleotide, which antisense oligonucleotide hybridizes to the pre-mRNA in the nucleus of the cell to create a duplex thereof under conditions which permit splicing, and wherein the antisense oligonucleotide blocks a member of the aberrant second set of splice elements so that the native intron is removed by splicing and the protein of interest is produced.

A second aspect of the present invention is a vector (e.g., a viral vector) useful for upregulating expression of a protein of interest (e.g., a native protein) in a cell, the cell containing a DNA encoding the protein, which DNA contains a mutation that causes downregulation of the protein by aberrant splicing in a pre-mRNA, wherein the DNA encodes the pre-mRNA; wherein the pre-mRNA contains a native intron having a first set of splice elements, which native intron is removed by splicing when the mutation is absent to produce a first mRNA encoding the protein; and wherein the pre-mRNA further contains an aberrant intron different from the native intron having a second set of splice elements, which aberrant intron is removed by splicing when the mutation is present to produce an aberrant second mRNA different from the first mRNA. The vector comprises a promoter operably associated with a nucleic acid sequence encoding a heterologous RNA (e.g., a small nuclear RNA), the heterologous RNA comprising a nuclear localization element joined to an antisense oligonucleotide, which antisense oligonucleotide hybridizes to the pre-mRNA in the nucleus of the cell to create a duplex thereof under conditions which permit splicing, and wherein the antisense oligonucleotide blocks a member of the aberrant second set of splice elements so that the native intron is removed by splicing and the protein of interest is produced.

The present invention is explained in greater detail in the drawings herein and the specification set forth below.

### BRIEF DESCRIPTION OF THE DRAWINGS

FIG. 1. Scheme of correction of aberrant splicing by modified U7 snRNA. Boxes—exons, lines—introns, short bars above and below exons—primers used in RT-PCR analysis. The dashed lines represent correct and aberrant splicing pathways. The modified U7 snRNA targeted to the IVS2-705 splice site (5′) is depicted under the pre-mRNA.

FIG. 2. Structure of U7 snRNA constructs. Wild-type U7 snRNA (heavy line) includes a stem-loop structure, the U7-specific Sm sequence (open box) and a sequence antisense to the 3′ end of histone pre-mRNA (stippled box). In anti-705 U7 snRNAs, the two sequences are replaced with the SmOPT sequence and with antisense sequences to the aberrant 3′ or 5 ′ splice sites in the β-globin gene, respectively. The promoter (prom.) and 3′ end forming (term.) regions are indicated. Short bars above and below the U7 construct represent primers used in PCR and RT-PCR analysis.

FIG. 3. Sequences of U7 snRNA constructs. The Sm binding site is boxed and the antisense sequences are underlined.

### DETAILED DESCRIPTION OF THE INVENTION

Nucleotide sequences are presented herein by single strand only, in the 5 ′ to 3′ direction, from left to right.

A. Intron-exon Splicing and Antisense Segments.

In nature, introns are portions of eukaryotic DNA which intervene between the coding portions, or “exons,” of that DNA. Introns and exons are transcribed into RNA termed “primary transcript, precursor to mRNA” (or “pre-mRNA”). Introns must be removed from the pre-mRNA so that the native protein encoded by the exons can be produced. The removal of introns from pre-mRNA and subsequent joining of the exons is carried out in the splicing process.

Introns are defined by a set of “splice elements” which are relatively short, conserved RNA segments that bind the various splicing factors which carry out the splicing reactions. Thus, each intron is defined by a 5′ splice site, a 3′ splice site, and a brand point situated therebetween. These splice elements are “blocked” as discussed herein when an antisense oligonucleotide either fully or partially overlaps the element, or binds to the pre-mRNA at a position sufficiently close to the element to disrupt the binding and function of the splicing factors which would ordinarily mediate the particular splicing reaction which occurs at that element (e.g., binds to the pre-mRNA at a position within 3, 6, 9, 12 or 15 nucleotides of the element to be blocked).

The mutation in the DNA and pre-mRNA may be either a substitution mutation or a deletion mutation that creates a new, aberrant, splice element. The aberrant splice element is thus one member of a set of aberrant splice elements that define an aberrant intron. The remaining members of the aberrant set of splice elements may also be members of the site of splice elements which define the intron. For example, if the mutation creates a new, aberrant 3′ splice site which is both upstream from (i.e., 5′ to) the native 3′ splice site and downstream from (i.e., 3′ to) the native branch point, then the native 5′ splice site and the native branch point may serve as members of both the native set of splice elements and the aberrant set of splice elements. In other situations, the mutation may cause native regions of the RNA which are normally dormant, or play no rule as splicing elements, to become activated and serve as splicing elements. Such elements are referred to as “cryptic” elements. For example, if the mutation creates a new aberrant mutation 3′ splice site which is situated between the native 3′ splice site and the native branch point, it may activate a cryptic branch point between the aberrant mutated 3′ splice site and the native branch point. In other situations, a mutation may create an additional, aberrant 5′ splice site which is situated between the native branch point and the native 5′ splice site and may further activate a cryptic 3′ splice site and a cryptic branch point sequentially upstream from the aberrant mutated 5′ splice site. In this situation, the native intron becomes divided into two aberrant introns, with a new exon situated therebetween. Further, in some situations where a native splice element (particularly a branch point) is also a member of the set of aberrant splice elements, it can be possible to block the native element and activate a cryptic element (i.e., a cryptic branch point) which will recruit the remaining members of the native set of splice elements to force correct splicing over incorrect splicing. Note further that, when a cryptic splice element is activated, it may be situated in either the intron or one of the adjacent exons. Thus, depending on the set of aberrant splice elements created by the particular mutation, the antisense oligonucleotide may be synthesized to block a variety of different splice elements to carry out the instant invention: it may block a mutated element, a cryptic element, or a native element; it may block a 5′ splice site, a 3′ splice site, or a branch point. In general, it will not block a splice element which also defines the native intron, of course taking into account the situation where blocking a native splice element activates a cryptic element which then serves as a surrogate member of the native set of splice elements and participates in correct splicing, as discussed above.

The length of the antisense oligonucleotide (i.e., the number of nucleotides therein) is not critical. In general, the antisense oligonucleotide is from 4, 6, 8, 10 or 12 nucleotides in length up to 20, 30, 50 or 100 nucleotides in length.

B. Viral Vectors.

Any viral vector can be used to carry out the present invention, including both DNA viruses and RNA viruses. All that is required is that the virus be capable of infecting the target cell or cells, and that the vector be capable of expressing the heterologous RNA in the cell. An oligonucleotide encoding the heterologous RNA used to carry out the present invention is inserted into the vector in operable association with and under the control of an appropriate promoter element, in accordance with known techniques. Examples of suitable viral vectors include, but are not limited to, retroviruses such as pLJ, adenoviruses, adeno-associated viruses, papovaviruses such as simian virus 40 and polyoma, etc. Numerous examples are known, including but not limited to those described in U.S. Pat. Nos. 5,240,846; 5,139,941; 5,252,479; 4,650,764; and 5,166,059 (the disclosures of which are incorporated by reference herein in their entirety.

Any suitable promoter element can be used in the viral vector to express the heterologous RNA in the target cell, so long as the promoter is operable in that cell. The promoter may conveniently be a small nuclear RNA promoter, as described below.

C. Oligonucleotides.

In addition to administration via a viral vector as described above, the oligonucleotides of the invention may be administered per se to the cells as described in U.S. Pat. No. 5,665,593 to Kole et al., the disclosure of which is incorporated by reference herein in its entirety. The oligonucleotide may be of any type, including natural and synthetic, but is preferably one which does not activate Rnase H. The oligonucleotide may be in the form of a physiologically and/or pharmaceutically acceptable salt. The oligonucleotide may be provided in a physiologically or pharmaceutically acceptable carrier, such as an aqueous carrier. The dosage of oligonucleotide will depend upon the particular method being carried out, and when it is being administered to a subject will depend on the disease, the condition of the subject, the particular formulation, the route of administration, etc. In general, intracellular concentrations of the oligonucleotide of from 0.05 to 50 uM, or more particularly 0.2 to 5 uM, are desired. For administration to a subject such as a human, a dosage of from about 0.01, 0.1 or 1 mg/Kg up to 50, 100, or 150 mg/Kg is employed.

D. Nuclear Localization Element.

The heterologous RNA includes a nuclear localization element (or “nuclear localization motif”) coupled to the antisense portion described above. Nuclear localization elements are known and may be provided from any suitable source, including natural and synthetic sources. For example, the nuclear localization element may be a site that binds to a protein that is found in or transported to the nucleus, such as an Sm binding site, a site that interacts with La protein, or a site that binds other snRNP-specific proteins, so that the heterologous RNA forms an snRNP complex (which complexes are very stable). Thus the nuclear localization element is typically of a size sufficient to assume a secondary structure, such as a stem-loop structure).

The nuclear localization element may be produced by combinatorial chemistry techniques, such as described in C. Grimm et al., In vivo selection of RNAs that localize in the nucleus, EMBO Journal 16(4), 793-806 (1997) and C. Grimm et al., In vivo selection of RNA sequences involved in nucleocytoplasmic RNA, Nucleic Acids Symposium Series (33): 34-6 (1995).

The nuclear localization element may be obtained from natural sources, including small nuclear RNA such as U1 and U6 RNA, which have been modified as carriers of antisense sequences that are designed to downregulate the targeted sequences (38-42). The anti sense oligonucleotide may be grafted onto the small nuclear RNA to alter the specificity thereof, allowing the use of the corresponding promoter element for the snRNA in the expression vector..

In general, the heterologous RNA, including the nuclear localization element and the antisense oligonucleotide portion, will be about 50 to 500 nucleotides in length.

E. Applications.

The present invention can be used in vitro to upregulate expression of a protein of interest at a desired period in time, for example after a growth phase, as described in U.S. Pat. No. 5,665,593 at column 4 line 61 to column 5 line 19, the disclosure of which is incorporated herein by reference. Administration can be carried out by any suitable means, such as by simply adding the vector to a growth medium containing the cells to be transformed.

The present invention can be used in vivo as a therapeutic agent in the treatment of disease involving aberrant splicing, such as β-thalassemia, α-thalassemia, Tay-Sachs syndrome, phylketonuria, certain forms of cystic fibrosis, etc. as described in U.S. Pat. No. 5,665,593 at column 5 lines 26-47, the disclosure of which is incorporated by reference herein in its entirety. Administration of the viral vector can be carried out by any suitable means, including parenteral injection (e.g., intraperitoneal, intraveneous, or intramuscular injection), oral administration, ihalation administration, etc. For administration the viral vector may be provided in a pharmaceutical carrier such as sterile saline solution.

The present invention is explained in greater detail in the following non-limiting examples.

### EXAMPLE

Disclosed herein is an approach that makes possible the stable expression of RNA antisense to aberrant thalassemic splice sites in β-globin pre-mRNA. This was accomplished by incorporating the anti-β-globin sequences into the gene for murine U7 small nuclear RNA (snRNA). U7 snRNA, in a complex with at least two U7 specific proteins and eight common Sm proteins (18), forms a ribonucleoprotein particle (U7 snRNP) which is involved in the processing of the 3′ end of histone pre-mRNAs (19-21). We show here that the insertion of appropriate antisense sequences into the U7 snRNA changed its function from a mediator of histone mRNA processing to an effector of alternative splicing of β-globin pre-mRNA. Stable transfection of cells expressing thalassemic β-globin gene with vectors carrying a modified U7 snRNA gene led to permanent correction of the splicing pattern of the β-globin pre-mRNA. This resulted in the accumulation of significant amounts of full length β-globin mRNA and the corresponding protein.

A. Materials and Methods

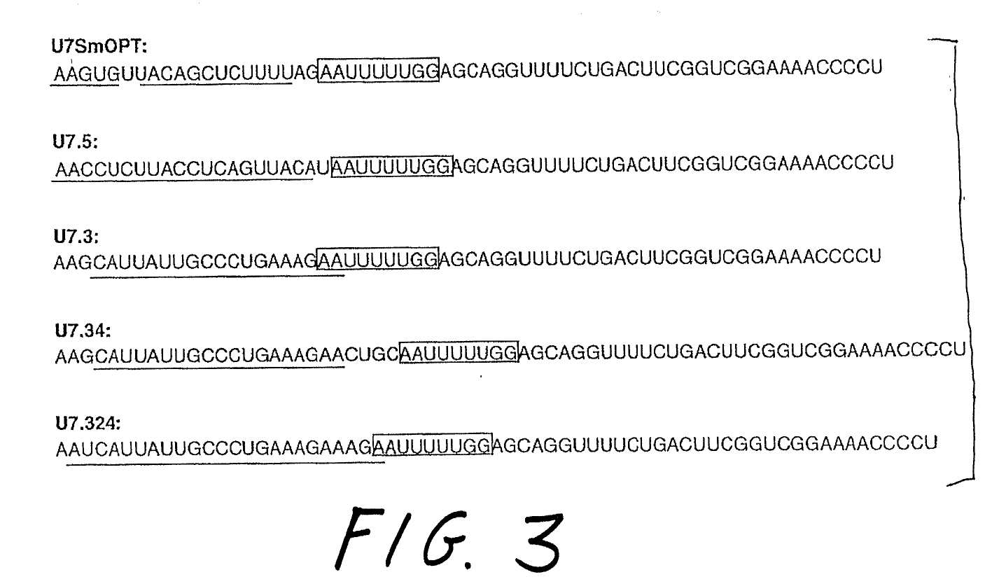

U7 snRNA constructs. The U7 Sm OPT plasmid carries the mouse U7 snRNA gene in which the U7-specific Sm binding site (AAUUUGUCUAG) was replaced with the consensus Sm sequence (AAUUUUUGGAG) (22). The U7 promoter and 3′ sequences are included in the construct. In U7.3, U7.5, U7.34 and U7.324 constructs, the natural 18-nucleotide sequence complementary to the 3′ processing site of histone pre-mRNAs was replaced (23, 24) with sequences complementary to either the 3′ or the 5′ splice sites activated by the IVS2-705 mutation (see FIG. 3).

Cell lines. The HeLa cell line carrying the thalassemic IVS2-705 human β-globin gene (25) and the cell lines stably expressing the modified U7 snRNAs were grown in S-MEM with 5% fetal calf and 5% horse sera. The latter cell lines were obtained by cotransfection of the HeLa IVS2-705 cells with a plasmid carrying a hygromycin resistance gene and a U7 snRNA expressing plasmid in the presence of Lipofectamine (8 μg/ml, Life Technologies) as recommended by the manufacturer. Stable transfectants were isolated after selection in media containing 250 μg/ml hygromycin.

Transient expression of modified U7 snRNA. For all experiments HeLa IVS2-705 cells were plated 24 hours before treatment in 24-well plates at 10⁵ cells per 2 cm² well. The cells were treated for 10 hrs with modified U7 plasmids (0.5, 1, 2 and 4 μg/ml) complexed with 8 μg/ml of Lipofectamine. Unless otherwise indicated, the cellular RNA or protein were isolated 24 hrs after the end of transfection.

RNA and DNA analysis. Total cellular RNA or DNA was isolated using TRI-Reagent (MRC, Cincinnati). 200 ng of RNA was analyzed by reverse transcription-PCR (RT-PCR) using rTth DNA polymerase as directed by the manufacturer (Perkin-Elmer). To maintain the linear concentration-dependent response, the PCR was carried out for 18 cycles (26) with addition of 0.2 μgCi of −[³²p]dATP to the PCR mixture. Correction of human β-globin pre-mRNA splicing was detected with forward and reverse primers spanning positions 21-43 of exon 2 and positions 6-28 of exon 3, respectively, in β-globin mRNA. Expression of modified U7 snRNA was assayed with forward:

(GCATAAGCTTAAGCATTATTGCCCTGAA)

and reverse:

(CGTAGAATTCAGGGGTTTTCCGACCGA)

primers; underlined nucleotides overlap with U7 sequences. RT-PCR products were separated on 7.5% nondenaturing polyacrylamide gels. The gels were dried and autoradiographed with Kodak BioMax film. For the control experiment (data not shown), 200 ng of chromosomal DNA was subjected to PCR using the same U7 specific primers.

Protein analysis. Hemin (10 μM, Fluka, Switzerland) treatment of transfected cells was in serum free medium for 4 hours immediately preceding the isolation of protein. Blots of proteins separated on a 10% Tricine-SDS polyacrylamide gel (27) were incubated with polyclonal affinity purified chicken anti-human hemoglobin IgG as primary antibody and rabbit anti-chicken horseradish peroxidase conjugated IgG as secondary antibody (Accurate Chemicals, Westbury, N.Y.). The blots were developed with an ECL detection system (Amersham).

Image processing. All autoradiograms were captured by DAGE MTI CCD72 video camera (Michigan City, Ind.) and the images were processed using NIH Image 1.57 and MacDraw Pro 1.0 software. The final figures were printed on Tektronix Phaser 550 printer. NIH Image 1.57 was also used for quantitation of the autoradiograms. Correctly spliced β-globin mRNA was quantified by densitometry of the autoradiograms with the results expressed as the percent correct product relative to the sum of the correct and aberrant products. The results were corrected to account for the higher [³²P]dAp content of the PCR product derived from aberrantly spliced mRNA than that from correctly spliced mRNA.

B. Results

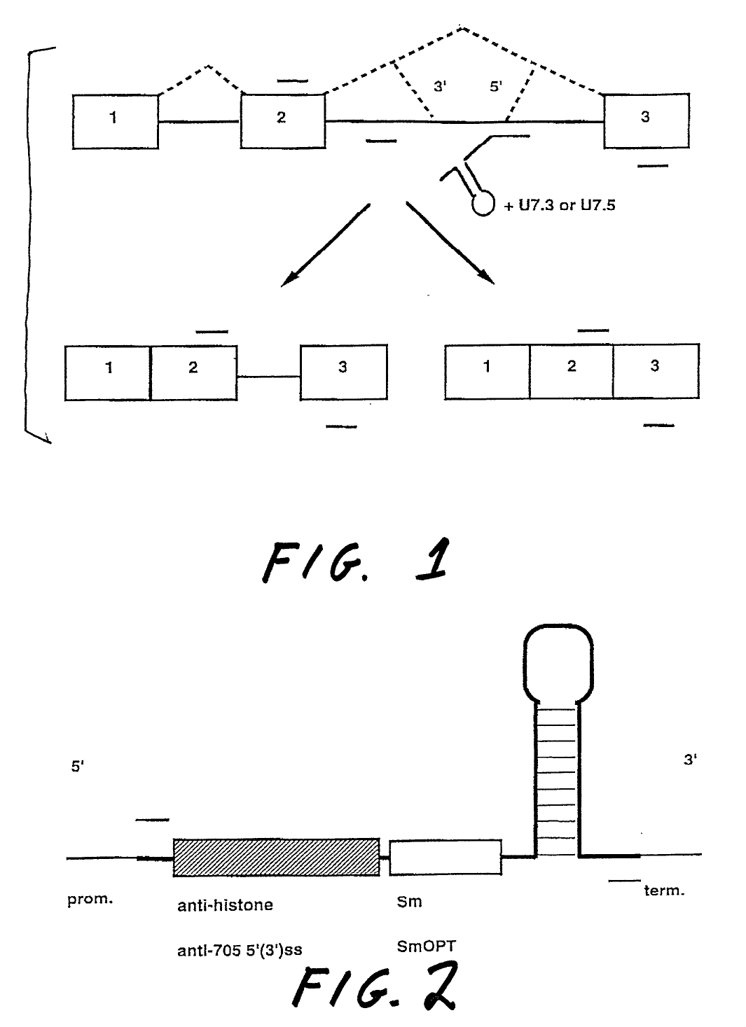

In the thalassemic IVS2-705 human β-globin gene, a T to G mutation at position 705 of intron 2 improves the match of the surrounding sequence to the consensus donor (5′) splice site (ACTGAT/GTAAGA to ACTGAG/GTAAGA; slash indicates the splice site). In the transcribed IVS2-705 pre-mRNA, the presence of this new 5′ splice site activates an acceptor (3′) splice site 126 nucleotides upstream, resulting in incorrectly spliced β-globin mRNA containing a fragment of the intron (FIG. 1). This fragment creates a premature stop codon resulting in a truncated β-globin polypeptide. Thus, in individuals homozygous for this mutation, the levels of the β-globin subunit of hemoglobin are drastically reduced, leading to β-thalassemia (28).

To improve the method of correction of splicing by antisense oligonucleotides (see Introduction) we have introduced into the U7 snRNA gene sequences encoding fragments antisense to the aberrant splice sites and used these constructs to transfect cells expressing the IVS2-705 pre-mRNA. It was anticipated that this approach will result in long term expression of antisense RNA. The choice of U7 snRNA and the design of the constructs (FIG. 2) as antisense carriers was based on several considerations.

The first 18 nucleotides of this 62 nucleotide-long RNA function as a natural antisense sequence by hybridizing with the so-called spacer element of histone pre-mRNA during its 3′ processing (29, 30). Thus, it seemed likely that upon replacement of the anti-histone sequence with a sequence complementary to aberrant splice sites in IVS2-705 pre-mRNA, the resulting U7 snRNA molecule would bind equally well to the new target sequences and correct aberrant splicing in a manner similar to antisense oligonucleotides.

Endogenous U7 snRNA is expressed at a low level, approximately 2-15×10³ molecules per cell. However, it was found that the expression level and the nuclear concentration of U7 snRNA could be significantly increased by converting the wild-type U7 Sm binding site (AAUUUGUCUCUAG) to the consensus Sm binding sequence derived from the major spliceosomal snRNPs (SmOPT, AAUUUUUGGAG) (31). Moreover, the SmOPT modification of U7 snRNA rendered the particle functionally inactive in histone pre-mRNA processing (22, 31). This potentially has two beneficial effects: (i) the target RNA, such as β-globin pre-mRNA, will not be cleaved by the histone 3′ end processing machinery; and (ii) due to the inability of U7 SmOPT particles to bind one or more U7-specific proteins (22), the RNA will not compete with endogenous U7 snRNP for potentially limiting U7 specific proteins. Finally, whereas the wild-type U7 snRNPs are sequestered in coiled bodies, those with the SmOPT modification are not (32) and therefore may be redirected to the sites of pre-mRNA splicing. Thus, the U7 gene with the SmOPT sequence was used to construct vectors expressing anti-705 U7 snRNAs (FIGS. 2 and 3) with the assumption that the increased nuclear concentration of the RNA and the lack of competition from the wild type molecule would improve its ability to block aberrant splice sites in IVS2-705 pre-mRNA.

The results of RT-PCR analysis of total RNA isolated 24 hours after transient transfection of a HeLa cell line expressing thalassemic IVS2-705 pre-mRNA with U7 constructs targeted to either of the aberrant splice sites were examined (data not shown). Both the U7 snRNA targeted to the aberrant 5′ splice site (data not shown) and the one targeted to the 3′ splice site (data not shown) corrected aberrant splicing of IVS2-705 pre-mRNA in a dose-dependent manner. Quantitative analysis of the results (See Materials and Methods) showed that at similar concentrations, the U7.3 and U7.5 RNAs corrected splicing to a similar level. At 2 μg/ml of DNA per 10⁵ cells, the level of correct splicing was approximately 50% for both constructs. Note that visualization of the correct and aberrant PCR products overestimates the amount of aberrantly spliced RNA since it contains approximately twice as many labeled adenosine nucleotides (see Materials and Methods) as the correct one. As expected, transfection of the cells with the vector expressing anti-histone U7 snRNA (U7SmOPT) had no effect on splicing of IVS2-705 pre-mRNA (data not shown), confirming the sequence specificity of the observed antisense effects.

In an attempt to improve correction of splicing, we have introduced two additional modifications into the U7.3 constructs. First, the antisense sequence was extended from 19 to 24 nucleotides (U7.324, FIG. 3) anticipating that the higher affinity of the longer sequence would increase the level of correct splicing. Second, since two of the nucleotides of the anti-globin sequence in U7.3 overlap with the Sm binding site (FIG. 3), it seemed possible that the bound Sm proteins might interfere with the antisense hybridization, reducing the correction of splicing. Hence, a 4 nucleotide spacer was inserted between the SmOPT element and the antisense sequence in construct U7.34 (FIG. 3).

Transfection of the IVS2-705 cells with the U7.324 plasmid led to a significant increase of correct splicing (data shown) relative to the unmodified U7.3 vector (data not shown). At 2 μg of vector DNA the level of correct splicing increased to 65% (data not shown). In contrast, addition of the 4 nucleotide spacer in the U7.34 construct (data not shown) or a ten nucleotide spacer (data not shown) had no beneficial effect on correction of splicing. It appears that extension of the antisense sequence improves the binding efficiency of the modified U7 snRNP whereas the Sm protein complex does not significantly interfere with the interactions between the 5′ end of the modified U7 snRNA and its target splice site.

Immunoblotting with polyclonal antibody to human hemoglobin of protein from cells transiently transfected with U7.324 by showed that the newly generated correctly spliced β-globin mRNA was translated into full length β-globin (data not shown). In agreement with RT-PCR results shown in FIG. 4A, cells with higher levels of correctly spliced β-globin mRNA contained increased amounts of full length β-globin (data not shown). However, at 4 μg plasmid (data not shown), the level of correctly spliced β-globin mRNA and the corresponding level of β-globin protein (data not shown) decreased. This was probably due to an incorrect charge ratio of the cationic lipid-DNA complex and the resultant poor uptake of the U7 plasmid (33). Clearly, the generation of the β-globin protein was due to the effect of U7.324 snRNA on IVS2-705 pre-mRNA splicing.

FIG. 5 shows the time course of the restoration of correct splicing of β-globin pre-mRNA after transient transfection of the IVS2-705 cell line with the U7.324 plasmid. RT-PCR analysis of the total RNA showed that a correction of splicing could be detected as early as 12 hours post-transfection (lane 6) and persisted through the 96 hour time point (lanes 7-9). Note that at 96 hours the transfected HeLa cells must have divided at least 3-4 times and yet the level of splicing correction remained essentially unchanged. This indicates that the expression of U7.324 snRNA, its stability, and the stability of the generated correctly spliced human β-globin mRNA are quite high. During the same time frame the treatment of cells with U7 Sm OPT control construct had no effect on splicing of IVS2-705 pre-mRNA (lanes 2-5).

Although in transient expression experiments the correction of splicing was evident for an extended period of time, the main advantage of the U7 vectors lies in their potential for permanent expression of antisense RNA and concomitant permanent correction of splicing. To test this possibility, stable cell lines were generated by cotransfecting IVS2-705 HeLa cells with the U7.324 vector and a plasmid carrying the hygromycin resistance marker. Analysis of hygromycin-resistant colonies showed that several clones corrected IVS2-705 pre-mRNA splicing, albeit at different levels (data not shown). In the most effective cell lines, the level of correction was 40 to 45% (data not shown).

Additional experiments provided evidence that the correction of splicing in the selected cell lines is a consequence of the expression of U7.324 snRNA. The U7 RNA levels were measured directly by RT-PCR of total cellular RNA with U7 specific primers (data not shown). The highest expression of U7.324 snRNA in cell line 705U7.324.4 correlates well with the highest level of correction observed in the same cell line. The expression of U7.324 RNA in the remaining cell lines (data not shown) is also commensurate with the correction of splicing (data not shown). PCR analysis of the DNA from the selected cell lines shows that the differences in the level of U7.324 RNA expression are most likely due to different copy numbers of the U7 genes (data not shown) as there is a correlation between the amounts of DNA amplification products and the levels of RNA expression and splicing correction. Finally, the possibility that the RT-PCR signal may have originated from genomic DNA contamination of the isolated RNA, was excluded by the absence of the U7-specific band (86 nucleotides) when the reverse transcription step was omitted from the RT-PCR protocol. The fact that PCR products were never detectable in the IVS2-705 parent cell line, which had not been transfected with the U7 vectors, attests to the sequence specificity of the assays and eliminates the possibility that the 86 nucleotide band was generated from endogeneous human U7 genes.

To ascertain that the stable transfection with U7 snRNA led not only to correction of splicing but also to stable expression of human β-globin, the protein lysates from another stable cell line 705U7.324.48 were assayed by immunoblotting. The results showed significant accumulation of full length β-globin protein (FIG, 7A, lane 3); accordingly the RT-PCR analysis showed that the level of splicing correction in this cell line was approximately 55%. Importantly, the stably transfected cells appear to have growth rates comparable to that of the wild type HeLa cells (data not shown), suggesting that the modified U7 snRNA is not toxic to the cells. We conclude that U7 snRNAs provide a specific and efficient mode of delivery of antisense sequences to the targeted splice sites.

C. Discussion

The expression of U7 snRNA, modified to hybridize to aberrant splice sites in IVS2-705 thalassemic human β-globin pre-mRNA, reduced the incorrect splicing of pre-mRNA and led to increased levels of the correctly spliced mRNA and β-globin protein. U7 constructs antisense to either the novel 5′ splice site created by the 705 mutation (U7.5) or the cryptic 3′ splice site activated in the aberrant splicing pathway (U7.3 and its derivatives) were effective at restoring correct splicing. The cryptic 3′ splice site is utilized by the splicing machinery in IVS2-654, IVS2-705 and IVS2-745 thalassemic pre-mRNAs (28). Thus, the U7.324 construct should be useful for correction of splicing in all three mutants. Levels of correction reached 65% in transient expression and 55% in stable cell lines transfected with U7.324. Restoration of β-globin to these levels in thalassemic patients would have been of therapeutic significance since transfusion therapy raises the hemoglobin to even lower levels yet improves the clinical status of the affected individuals (28).

The ability to generate cell lines in which the genetic defect that leads to incorrect splicing is by-passed and continuous production of a correct gene product is restored, is highly encouraging. These results suggest a possibility of gene therapy based on the antisense concept. The patients' bone marrow, in particular the erythroblasts and possibly the stem cells, could be transfected ex-vivo with the antisense U7 vectors and reimplanted. Even if the expression of the U7 snRNA were short lived, either due to lack of transfection of stem cells or to promoter shut-off, both being common problems in the expression of transgenes (34, 35), the results may be relatively long lasting. This is because correction of β-globin pre-mRNA splicing driven by antisense U7 snRNA should increase the production of β-globin and reduce the imbalance between the and β subunits of hemoglobin, consequently improving the survival of erythroblasts and promoting the maturation of erythrocytes. Since the life span of erythrocytes is approximately 120 days (36), the treated cells should persist in the blood stream for an extended period of time.

The possibility of overexpression and/or inappropriate expression of the transfected gene constitutes serious concerns in gene therapy. In fact, overexpression of the β-globin transgene may lead to a new imbalance between - and β-globin subunits and, conceivably, to symptoms of -thalassemia. In this context, the correction of splicing by antisense U7 molecules offers an advantage since the β-globin subunits may at best reach the wild type levels. Furthermore, even if the U7 snRNAs were inappropriately expressed in different cell types, their effects are expected to be limited only to cells that express the target sequence, β-globin pre-mRNA, i.e to nucleated erythroblasts. The sequence specificity of the effect of U7 snRNAs targeted to the splice sites is substantiated by the negative results seen with the control U7SmOPT snRNA. It is further reinforced by the finding that the GenBank database of human sequences contains no sequence other than human β-globin intron 2 that corresponds to the 5′- and 3′- splice sites, even allowing for two mismatches.

For repair of a splicing mutation at the RNA level, it would be optimal to obtain high levels of expression of antisense RNA in the nucleus, where both expression of target pre-mRNAs and splicing occur. Using U7 snRNA as an antisense carrier guarantees its nuclear localization, since the U7 snRNA will be transported from the cytoplasm to the nucleus in a manner similar to other Sm-type snRNAs. Due to their small size, secondary structure and tight interactions with common Sm and other snRNP-specific proteins (37), the snRNAs, or rather their snRNP complexes, are very stable. In clinical applications the above properties would reduce the frequency of patient treatment. The modification of wild-type U7 snRNA to SmOPT, which was shown to increase its stability and nuclear uptake, in conjunction with its constitutive expression (30), clearly provided sufficient concentrations of the RNA to ensure efficient binding to the targeted splice sites and correction of splicing.

Other snRNAs can also provide convenient delivery agents for antisense therapeutics. Both U1 and U6 RNA have been modified as carriers of antisense sequences designed to downregulate the targeted sequences (38-42). U1 snRNA appears to be a particularly attractive candidate since it is known to bind to its target sequences, the 5′ splice sites, via a base pairing mechanism. However, preliminary experiments showed that although a modified, transiently transfected U1 snRNA was efficiently transcribed, accounting for 25 to 30% of the total U1 RNA, its effect on splicing of the targeted adenovirus E1A or rabbit β-globin pre-mRNAs was minor (38). This may have been due to unstable binding of the 9 nucleotide antisense sequence of the modified U1 RNA to its target, the inaccessibility of the target, or to out-competition by wild-type U1 RNA. Interestingly, the anti-705 U7snRNA with its 24 nucleotide antisense sequence was expressed at the level equal to that of endogenous U7 snRNA (Reber and Shumperli, data not shown). That, and the concomitant lack of competition between the two molecules, are likely to be responsible for the successful alteration of splicing reported here.

Since up to 15% of all point mutations in genetic diseases have been estimated to result in defective splicing (43), our approach may not be limited to thalassemic mutations. Furthermore, the same approach can be used to modify normal splicing patterns of constitutively and alternatively spliced pre-mRNAs resulting in changes in gene expression. Apart from the potential clinical applications, the ability to permanently modify splicing patterns of specific pre-mRNA may also prove useful in studies on the control of gene expression.

### References

The following references are cited herein, by the reference numbers indicated.

1. Tolstoshev, P. (1993) Ann. Rev. Pharm. and Tox. 33, 573-596.

2. Fenjves E S. Schwartz P M. Blaese R M. Taichman L B. (1997) Hum. Gene Ther. 8, 911-917.

3. Knowles, M. R., Hohneker, K. W., Zhou, Z., Olsen, J. C., Noah, T. L., Hu, P. C., Leigh, M. W., Engelhardt, J. F., Edwards, L. J., Jones, K. R. et al. (1995) New Engl. J Med. 333, 823-831.

4. Acsadi, G., Dickson, G., Love, D. R., Jani, A., Walsh, F. S., Gurusinghe, A., Wolff, J. A., Davies, K. E. (1991) Nature 352, 815-818.

5. Dunbar, C., Kohn, D. (1996) Hum. Gene Ther. 7, 231-253.

6. Byun, J., Kim, S H., Kim, J M., Yu, S S., Robbins, P D., Yim, J., Kim, S. (1996) Gene Ther. 3, 780-788.

7. Shi, Q., Wang, Y., Worton, R. (1997) Hum. Gene Ther. 8, 403-410.

8. Zhou, S. Z., Li, Q., Stamatoyannopoulos, G., Srivastava, A., (1996) Gene Ther. 3, 223-229.

9. Ellis, J., Pasceri, P., Tan-Un, K.C., Wu, X., Harper, A., Fraser, P., Grosveld, F. (1997) Nucleic Acids Res. 25, 1296-1302.

10. Sadelain, M., Wang, C. H., Antoniou, M. Grosveld, F., Mulligan, R.C. (1995) Proc. Natl. Acad. Sci. USA 92, 6728-6732.

11. Cole-Strauss, A., Yoon, K., Xiang, Y., Byrne, B. C., Rice, M. C., Gryn, J., Holloman, W. K., Kmiec, E. B. (1996) Science 273, 1386-1389.

12. Perrine S P. Miller B A. Faller D V. Cohen R A. Vichinsky E P. Hurst D. Lubin B H. Papayannopoulou T. (1989) Blood 74, 454-459.

13. Charache, S., Barton, F. B., Moore, R. D., Terrin, M. L., Steinberg, M. H., Dover, G. J., Ballas, S. K., McMahon, R. P., Castro, O., Orringer, E. P. (1996) Medicine 75, 300-326.

14. Sher, G. D., Ginder, G. D., Little, J., Yang, S., Dover, G. J., Olivieri, N. F. (1995) New Engl. J. Med. 332, 1606-1610.

15. Collins, A. F., Pearson, H. A., Giardina, P., McDonagh, K. T., Brusilow, S. W., Dover, G, J. (1995) Blood 85, 43-49.

16. Sierakowska, H., Sambade, M. J., Agrawal, S., Kole, R. (1996) Proc. Natl. Acad. Sci. USA 93, 12840-12844.

17. Dominski, Z., Kole, R. (1993) Proc. Natl Acad. Sci. USA 90, 8673-8677.

18. Smith, H. O., Tabiti, K., Schaffner, G., Soldati, D. Albrecht, U., Birnstiel, M. L. (1991) Proc. Natl. Acad Sci. USA 88, 9784-88.

19. Galli, G., Hofstetter, H., Stunnenberg, H. G., Bimstiel, M. L. (1983) Cell 34, 823-828.

20. Birchmeier, C., Schümperli, D., Sconzo, D., Bimstiel, M. L. (1984) Proc. Natl. Acad. Sci. USA 81, 1057-1061.

21. Bimstiel, M. L. and Schaufele, F. (1988) in Structure and function of major and minor small nuclear ribonucleoprotein particles, ed. Bimstiel, M. L., (Berlin) pp. 155-182.

22. Stefanovic, B., Hackl, W., Lührmann, R., Schümperli, D. (1995) Nucleic Acids Res. 23, 3141-3151.

23. Jones, D. H. and Howard, B. H. (1991) Biotechniques 10, 62-66.

24. Jones, D. H. and Winistorfer, S. C. (1992) Biotechniques 12, 528-535.

25. Sierakowska, H. Montague, M., Agrawal, S., and Kole, R. (1997) Nucleotides and Nucleosides 16, 1173-1182.

26. Chen, I. T., Chasin, L. A. (1993) Mol. Cell Bio. 13, 289-300.

27. Schagger, H. and von Jagov, G. (1987) Anal. Biochem. 166, 368-379.

28. Schwartz, E., and Benz, E. J. (1991) in Hematology: basic principles and practice, eds. Hoffman, R., Benz, E. J., Shattil, S. J., Furie, B., Cohen, H. J. (Churchill Livingstone, New York) pp. 368-392.

29. Bond, U., Yario, T. A., Steitz, J. (1991) Genes Dev. 5, 1709-1722.

30. Spycher, C., Streit, A., Stefanovic, B., Albrecht, D. Konig, T. H., Schümperli, D. (1994) Nucleic Acids Res. 22, 4023-4030.

31. Grimm, C., Stefanovic, B., Schümperli, D. (1993) EMBO J. 12, 1229-1238.

32. Wu, C. H., Murphy, C., Gall, J. G. (1996) RNA 2, 811-823.

33. Hawley-Nelson, P., Ciccarone, V., Gebeyehu, G., Jessee, J., Felgner, P.L. (1993) Focus (Rochester, N.Y.) 15, 73-79.

34. Page S. M., Brownlee G. G. (1997) J Invest. Derm. 109, 139-145.

35. Palmer, T. D., Rosman, G. J., Osborne, W. R., Miller, A. D. (1991) Proc Natl Acad Sci USA 88, 1330-1334.

36. Eadie, G. S., Brown, I. W., Curtis, W. G. (1955) J. Clin. Invest. 34, 629.

37. Lührmann, R., Kastner, B., Bach, M. (1990) Biochim. Biophys. Acta 1087: 6255-6270.

38. Yuo, C., Weiner, A. M. (1989) Mol. Cell. Bio. 9: 3429-3437.

39. Montgomery, R. A., Dietz, H. C. (1997) Hum. Molec. Gen. 6, 519-525.

40. Good, P. D., Krikos, A. J., Li, S. X. L., Bertrand, E., Lee, N. S., Giver, L., Ellington, A., Zaia, J. A., Rossi, J. J., Engelke, D. R. (1997) Gene Ther. 4, 45-54.

41. Liu, D., Donegan, J., Nuovo, G., Mitra, D., Laurence, J. (1997) J. Virol. 71, 4079-4085.

42. Noonberg, S. B., Scott, G. K., Garavoy, M. R., Benz, C. C., Hunt, C. A. (1994) Nuc. Acids Res. 22, 2830-2836.

43. Krawczak, M., Reiss, J., Cooper, DN. (1992) Hum. Gen. 90, 41-54.

The foregoing is illustrative of the present invention, and not to be construed as limiting thereof. The invention is defined by the following claims, with equivalents of the claims to be included therein.

We claim:

1. A method of upregulating expression of a protein of interest in a cell, said cell containing a DNA encoding said protein, which DNA contains a mutation that causes downregulation of said protein by aberrant splicing in a pre-mRNA,
wherein said DNA encodes said pre-mRNA;
wherein said pre-mRNA contains a native intron having a first set of splice elements, which native intron is removed by splicing when said mutation is absent to produce a first mRNA encoding said protein;
and wherein said pre-mRNA further contains an aberrant intron different from said native intron having a second set of splice elements, which aberrant intron is removed by splicing when said mutation is present to produce an aberrant second mRNA different from said first mRNA;
said method comprising:
administering to said cell a heterologous oligonucleotide, said heterologous oligonucleotide comprising a nuclear localization element joined to an antisense oligonucleotide, which antisense oligonucleotide hybridizes to said pre-mRNA in the nucleus of said cell to create a duplex thereof under conditions which permit splicing, and wherein said antisense oligonucleotide blocks a member of said aberrant second set of splice elements so that said native intron is removed by splicing and said protein of interest is produced.

2. A method according to claim 1, wherein said administering step is carried out in vivo.

3. A method according to claim 1, wherein said administering step is carried out in vitro.

4. A method according to claim 1, wherein said administering step is carried out by administering a vector that expresses said heterologous oligonucleotide in said cell.

5. A method according to claim 4, wherein said vector is a viral vector.

6. A method according to claim 5, wherein said heterologous oligonucleotide comprises RNA.

7. A method according to claim 1, wherein said administering step is carried out by administering an exogeneous oligonucleotide to said cell.

8. A method according to claim 1, wherein said nuclear localization element forms an snRNP complex in said cell.

9. A method according to claim 1, wherein said nuclear localization element comprises small nuclear RNA.

10. A method according to claim 9, wherein said nuclear localization element comprises U1 or U6 RNA.

11. A vector useful for upregulating expression of a protein of interest in a cell, said cell containing a DNA encoding said protein, which DNA contains a mutation that causes downregulation of said protein by aberrant splicing in a pre-mRNA,
wherein said DNA encodes said pre-mRNA;
wherein said pre-mRNA contains a native intron having a first set of splice elements, which native intron is removed by splicing when said mutation is absent to produce a first mRNA encoding said protein;
and wherein said pre-mRNA further contains an aberrant intron different from said native intron having a second set of splice elements, which aberrant intron is removed by splicing when said mutation is present to produce an aberrant second mRNA different from said first mRNA;
said vector comprising:
a promoter operably associated with a nucleic acid sequence encoding a heterologous RNA, said heterologous RNA comprising a nuclear localization element joined to an antisense oligonucleotide, which antisense oligonucleotide hybridizes to said pre-mRNA in the nucleus of said cell to create a duplex thereof under conditions which permit splicing, and wherein said antisense oligonucleotide blocks a member of said aberrant second set of splice elements so that said native intron is removed by splicing and said protein of interest is produced.

12. A vector according to claim 11, wherein said vector is a viral vector.

13. A vector according to claim 11, wherein said nuclear localization element forms an snRNP complex in said cell.

14. A vector according to claim 11, wherein said nuclear localization element comprises small nuclear RNA.

15. A vector according to claim 11, wherein said nuclear localization element comprises U1 or U6 RNA.

16. An oligonucleotide useful for upregulating expression of a protein of interest in a cell, said cell containing a DNA encoding said protein, which DNA contains a mutation that causes downregulation of said protein by aberrant splicing in a pre-mRNA,
wherein said DNA encodes said pre-mRNA;
wherein said pre-mRNA contains a native intron having a first set of splice elements, which native intron is removed by splicing when said mutation is absent to produce a first mRNA encoding said protein;
and wherein said pre-mRNA further contains an aberrant intron different from said native intron having a second set of splice elements, which aberrant intron is removed by splicing when said mutation is present to produce an aberrant second mRNA different from said first mRNA;
said oligonucleotide comprising a nuclear localization element joined to an antisense oligonucleotide, which antisense oligonucleotide hybridizes to said pre-mRNA in the nucleus of said cell to create a duplex thereof under conditions which permit splicing, and wherein said antisense oligonucleotide blocks a member of said aberrant second set of splice elements so that said native intron is removed by splicing and said protein of interest is produced.

17. An oligonucleotide according to claim 16, wherein said nuclear localization element forms an snRNP complex in said cell.

18. An oligonucleotide vector according to claim 16, wherein said nuclear localization element comprises small nuclear RNA.

19. An oligonucleotide according to claim 16, wherein said nuclear localization element comprises U1 or U6 RNA.

20. An oligonucleotide according to claim 16, wherein said oligonucleotide is about 50 to 500 nucleotides in length.
